(19) United States
(12) Patent Application Publication
(10) Pub. No.: US 2002/0177763 A1
(43) Pub. Date: Nov. 28, 2002

(54) Integrated lancets and methods

(76) Inventors: David W. Burns, San Jose, CA (US); John T. Dangtran, San Jose, CA (US); John F. Di Cristina, Acton, MA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/863,710

(22) Filed: May 22, 2001

(51) Int. Cl. A61B 5/05; G01N 33/48

(52) U.S. Cl. 600/345; 600/347; 422/68.1

(57) ABSTRACT

An integrated lancet/biosensor comprised of a small, sharply tapered silicon lance and a body region containing active devices, passive trimming structures and features for accurate assembly. The lancet contains a series of electrodes covered with a specialized reagent to provide an output signal proportional to the quantity of the specific material in the blood or other bodily fluid. Trimming and amplification of the electrical signal are achieved with front-end electronic circuitry fabricated on the lancet body. The lancet is manufactured using integrated circuit fabrication techniques and micromachining techniques, and assembled into a disposable probe tip that contains a lead frame and pins. The probe tip may be attached to a pencil-shaped meter body that has additional circuitry for references, compensation, display drivers and external communication. Various embodiments are disclosed.

### BACKGROUND OF THE INVENTION

1. Field of the Invention

The present invention relates to the field of biosensors.

2. Prior Art

Biosensors of various kinds to measure various biological quantities are well known in the prior art. Such sensors generally are available in two types. One type, used for individual measurements, requires that the bodily fluid to be tested be withdrawn from the body and applied to some form of test strip or sensor external to the body. Common glucose sensors used by diabetics are of this type, requiring the puncture of the skin to withdraw blood or to induce some bleeding to provide the blood sample for the sensor. Another type of sensor, used for continuous monitoring during surgical procedures, requires insertion of the sensor into the body, such as into the blood stream, or diversion of the flow of the fluid over the sensor, for proper operation. This is more invasive that the puncturing of the skin by a needle or sharp point to withdraw enough fluid for test purposes. The present invention is an integrated lancet/biosensor that measures the biological quantity within the body, but is less invasive than even a needle or a commonly used sharp point.

### BRIEF SUMMARY OF THE INVENTION

The invention disclosed herein is a structure for the measurement of biological quantities such as blood glucose. The invention combines integrated circuit fabrication techniques with micromachining techniques to produce an integrated lancet/biosensor. The integrated lancet is comprised of a small, sharply tapered silicon lance and a body region containing active devices, passive trimming structures and features for accurate assembly. The lance contains a series of electrodes covered with a specialized reagent to provide an output signal proportional to the quantity of the specific material in the blood or other bodily fluid. Trimming and amplification of the electrical signal are achieved with front-end electronic circuitry fabricated on the lancet body. The lancet is assembled into a disposable probe tip that contains a lead frame and pins. The probe tip may be attached to a pencil-shaped meter body that has additional circuitry for references, compensation, display drivers and external communication.

Various embodiments are disclosed.

### BRIEF DESCRIPTION OF THE DRAWINGS

FIGS. 1a through 1d illustrate an embodiment of integrated lancet of the present invention.

FIG. 2 is an illustration showing the lancet of FIG. 1 molded in disposable probe tip.

FIG. 3 illustrates an alternate embodiment of the invention, specifically a straight-body version of the integrated lancet.

FIG. 4 illustrates the wafer fabrication of lancets wherein a large number of lancets may be fabricated on a single wafer of semiconductor material, typically a silicon wafer.

FIGS. 5a through 5d present an exemplary process flow for fabrication of the integrated lancet of the present invention.

FIGS. 6a through 6c illustrate lancet surface capillaries micromachined into the lancet tip to aid in the transport of bodily fluids to the reaction sites during use.

FIGS. 7a through 7b illustrate the used of tape automated bonding and transfer molded disposable tip, wherein the pads on the integrated lancet are bumped and soldered directly to a lead frame with no wirebonds.

FIG. 8 illustrates a hand held meter assembly wherein the disposable probe tip is inserted into a meter body containing a display, alarm and external connection port.

FIGS. 9a through 9c illustrate an integrated lancet using four connection pads.

FIGS. 10a through 10c illustrate an integrated lancet secured to a plastic probe tip, wire bonded to the molded lead frame and over molded to form a disposable probe tip in accordance with the present invention.

FIGS. 11a and 11b illustrates a hand held meter and connector wherein the meter body houses batteries, an LCD display, an IrDA port and an internal PC board, the connector allowing mating of the disposable probe tip to the meter.

FIGS. 12a through 12d illustrate exemplary lancet biosensor circuits.

FIG. 13 presents an exemplary biosensor meter schematic.

FIG. 14 presents an exemplary ASIC (application specific integrated circuit) schematic for the ASIC 70 of FIG. 13.

FIG. 15 is an exemplary generalized flow diagram for process, test and assembly of lancets in accordance with the present invention.

### DETAILED DESCRIPTION OF THE PREFERRED EMBODIMENT

The integrated lancet/sensors of the present invention (often simply referred to as a lancet for convenience) is a MEMS (micro-electro-mechanical-system) device for an accurate, low cost, convenient and low pain approach to the measurement of blood glucose and other bodily constituents. A combination of micromachining, integrated circuit (IC) processing and package/assembly methods are used to produce a micro-lance with sensor electrodes, integrated electronics, wire-bonded lead frame and plastic over-molding, in the preferred embodiment for blood glucose measurement. The preferred embodiment includes a micromachined lancet, plate reaction sites at the tip, minimal blood volume, no enclosed capillary, fast response, on-chip integrated electronics, combination lead frame/connector pins and an over-molded plastic housing.

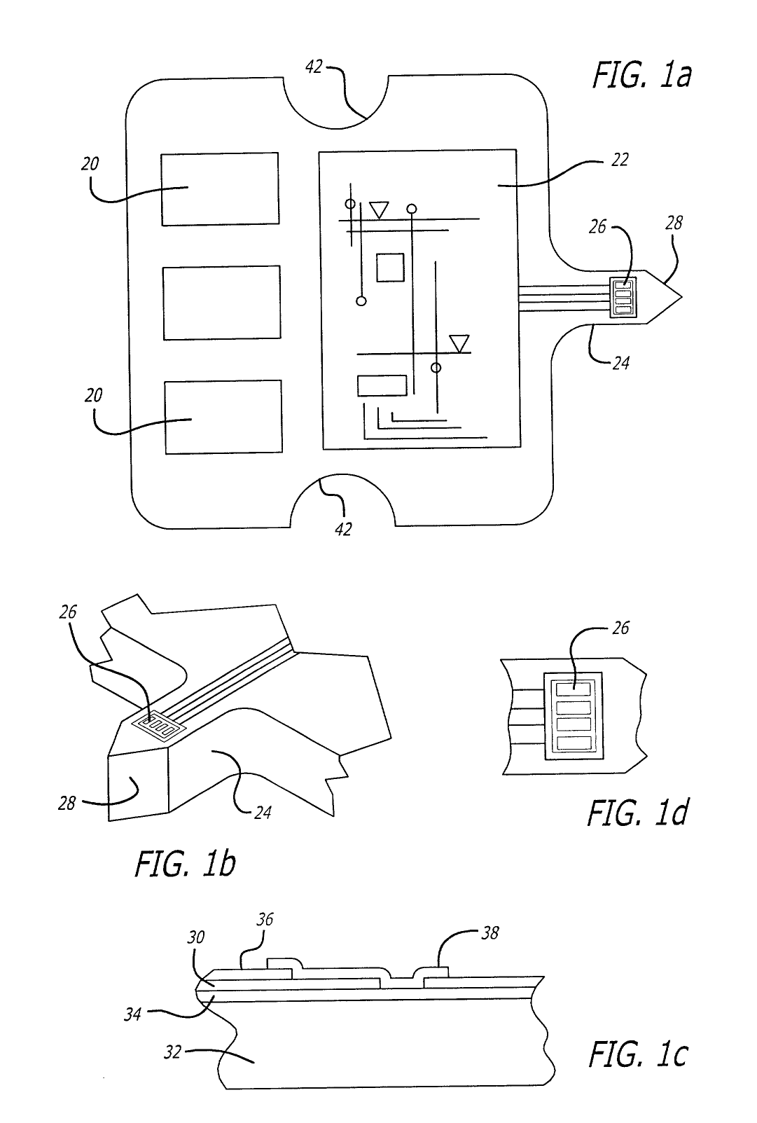

One embodiment of the integrated lancet is shown in FIG. 1a. The lancet is typically formed on a silicon substrate, and is characterized by bond pads 20, in this embodiment 3 bond pads, integrated electronics 22, a needle shank 24, reaction sites 26 and a sharp lance tip 28. The device can be less than ½ millimeter on a side, including the lance tip, depending on the level of integration of electronics desired in the lancet. In that regard, it is preferable to at least provide buffer circuitry on the lancet, though higher level signal processing may be included as desired. In such a configuration, the body containing the integrated electronics 22 and bond pads 20 may be approximately 400 μm on a side, the needle shank 24 approximately 80 μm long and the lance tip 28 approximately 20 μm long. A three-dimensional view of the lancet tip 28 is shown in FIG. 1b. The lance tip may have a thickness between 100 micrometers to over 625 micrometers, depending on the strength requirements of the lancet.

A cross-sectional view of the lancet is shown in FIG. 1c, showing an electrode metal 30 (such as platinum) isolated from the silicon substrate 32 by an oxide dielectric 34, and coated with a passivation layer 36 such as silicon nitride. A gel or reagent 38 is coated over the electrodes to provide specificity to the material being measured, such as glucose oxidase for the measurement of blood glucose.

A top view of the electrode area is shown in FIG. 1d. In this embodiment, a Kelvin arrangement of four electrodes is used. The two outer electrodes provide a predetermined current through the reagent, by which the resistivity of the reagent may be determined by measuring the voltage across the two center electrodes by a high input impedance amplifier. Of course other measurement methods using two or three electrodes can be applied for measuring current or chemical potential at the reaction site, as desired.

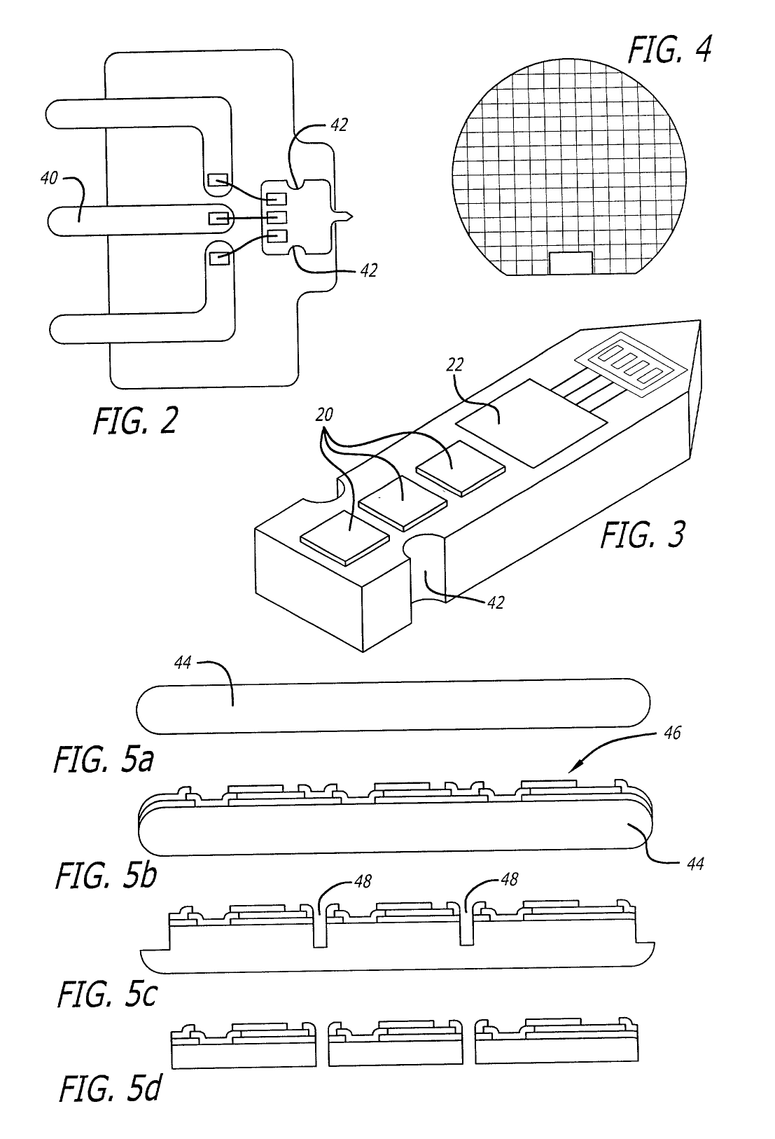

In the exemplary embodiment, the integrated lancet is molded into a plastic housing containing a lead frame assembly 40, as shown in FIG. 2. The integrated lancet preferably contains micromachined detents 42 to enhance adherence between the plastic and the silicon. The detents can be semicircular, triangular, barbed or other shapes as desired.

A straight-body version of the lancet is shown in FIG. 3, with the bonding pads 20 oriented along the central axis of the lancet. Either version, or further alternates of the lancet can be batch fabricated in large arrays from silicon wafers, as illustrated in FIG. 4.

A simplified process flow for the integrated lancet is illustrated in FIGS. 5a through 5d, where standard silicon wafers 44 (n-type or p-type) are fabricated with integrated electronics and additional trimming circuitry, generally indicated by the numeral 46 (FIGS. 5a and 5b). The IC processing can be bipolar, CMOS or BiCMOS to meet the performance, power and cost requirements of the on-chip circuitry. A single photomask is then used to form the irregular-shaped body and lancet tip by using a photoresist mask and etching deep trenches 48 into the silicon wafer (i.e., 150 μm deep) as shown in FIG. 5c. After removal of the photoresist mask, a backside grinding or etching operation is used to separate the lancets, as shown in FIG. 5d. A backing wafer or carrier (not shown) is used on the front surface of the wafer during this operation to hold the lancets. Other fabrication techniques may be used as desired.

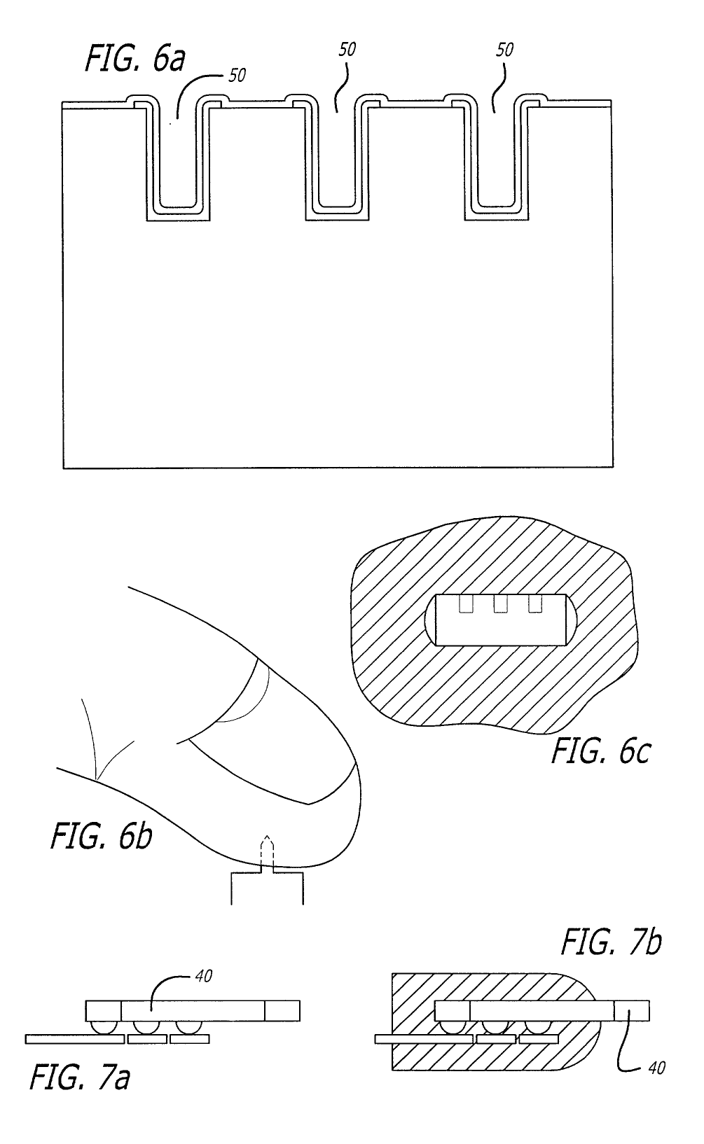

An additional micromachining step can be incorporated into the lancet to improve the ability of bodily fluids to reach the reagent and electrodes. Narrow three-walled capillaries may be formed in the lancet tip to guide biological fluids to the electrodes in cases where skin and other epidermal material obscures the electrodes. The microcapillaries 50 are shown in FIG. 6a, with the lancet tip penetrating the skin in FIG. 6b (not to scale), resulting in possible obscuring of the electrodes (FIG. 6c). The microcapillaries can have an exemplary aspect ratio (depth to width) greater than 2:1 for effective fluidic flow.

The integrated lancet can have bumped (plated) reflow pads that orient directly with a custom lead frame assembly as shown in FIG. 7a for bonding by reflow. The assembly is overmolded with plastic prior to excising the lead frame, as shown in FIG. 7b.

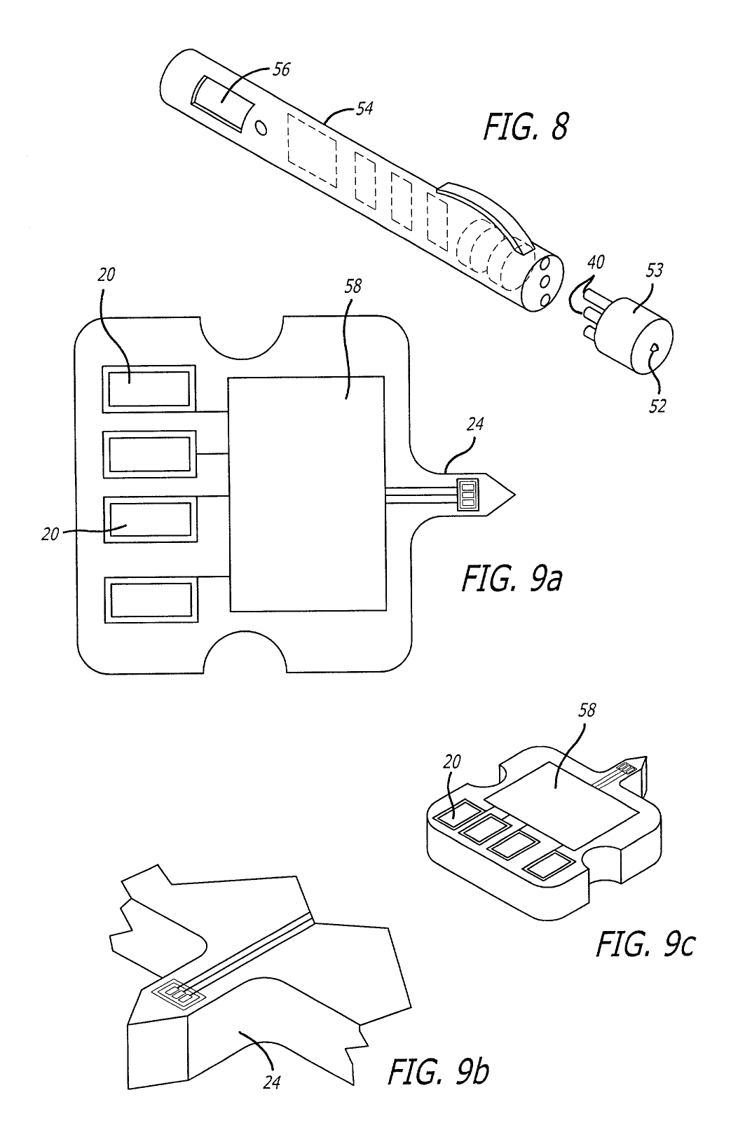

The integrated lancet and plastic housing 53 form a disposable tip for single use measurement of bodily fluids, as shown in FIG. 8. The lead frame 40 is configured to have three or four pins, which allow the probe tip 52 to be plugged into a meter body. A cylindrical, pencil-shaped meter body 54 is shown in FIG. 8, containing a PC board, amplification circuitry, digital signal processing circuitry, internal memory and clock circuitry, circuitry for external communication such as an IrDA interface, display drivers, an LCD display 56, batteries and a convenient pen clip, for processing the signal from the probe tip 52 and displaying the result and/or communicating with other equipment by a wired or wireless connection, such as RF or infrared communication.

A four-pad version of the lancet is depicted in FIGS. 9a and 9c. The top view (FIG. 9a) shows the four pads 20, location of the amplification and trim circuitry 58, triple electrodes and lancet tip 24 with typical dimensions. FIG. 9b shows a three-dimensional view of the lancet tip, with the entire structure shown in FIG. 9c.

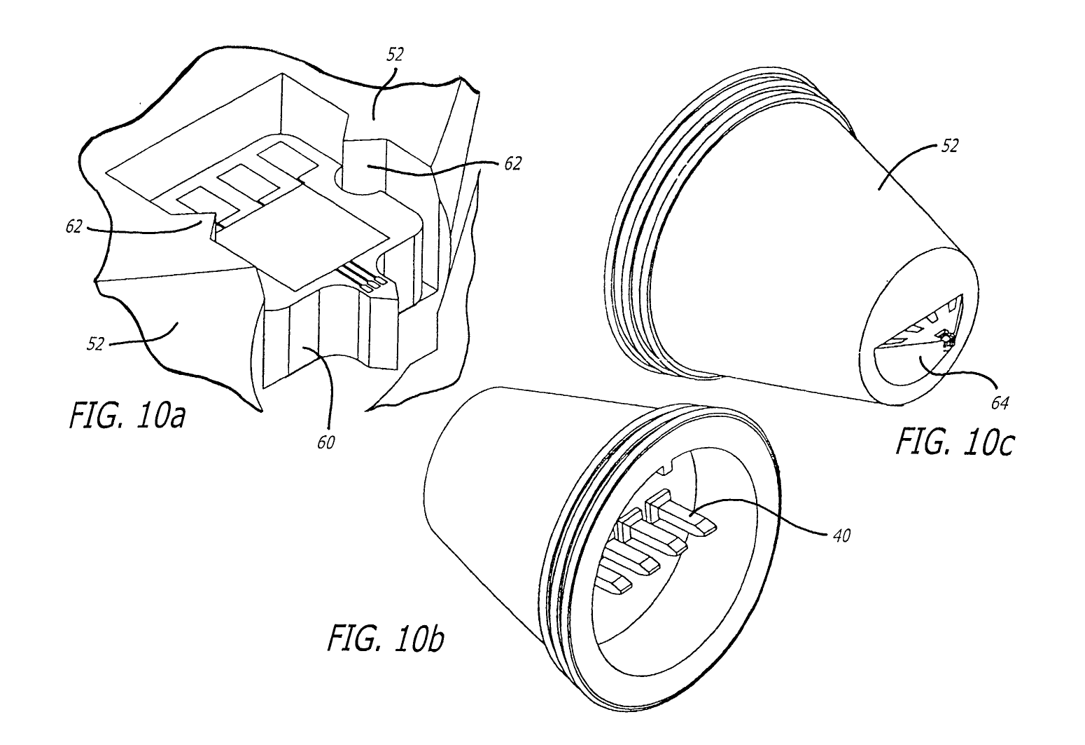

A lancet 60 assembled into the plastic probe tip 52 is shown in FIG. 10a, with protrusions 62 in the plastic housing and complementary recesses in the lancet 60 used to firmly hold the silicon lancet. The backside of the disposable probe tip with lead frame pins 40 is visible in FIG. 10b, along with a magnified frontal view showing the placement of the lancet in FIG. 10c. The conical tip 64 is shown in the cutaway view to reveal the lead frame and bond pads.

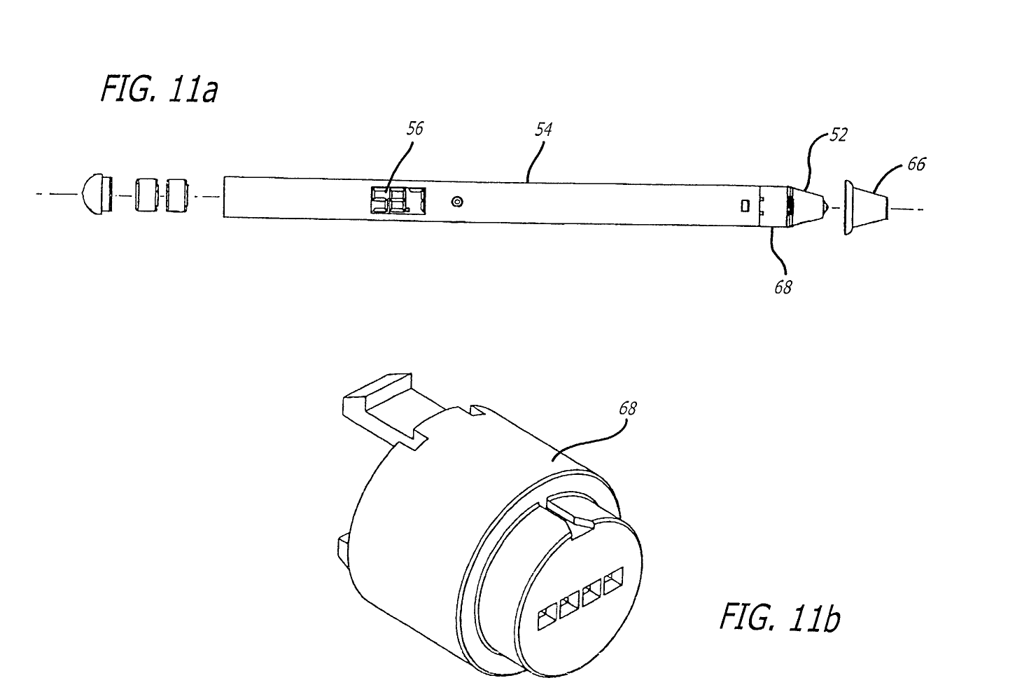

A hand-held meter body 54 containing the interface and communication electronics, batteries and display 56 is also shown in FIG. 11a, along with a cover 66 for the disposable probe 52. A connector socket 68 is shown in detail in FIG. 11b. The meter body in this exemplary embodiment is approximately 5.3 inches long and ⅜ inch in diameter, with the disposable probe tip being on the order of 0.385 inches long.

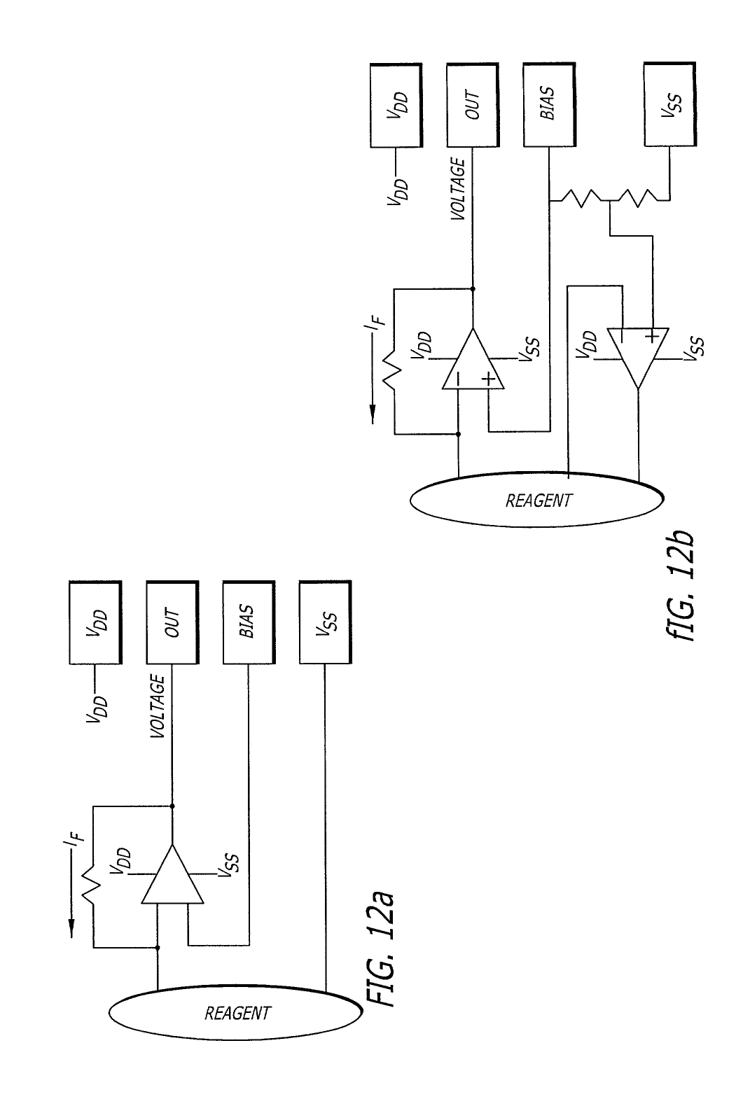

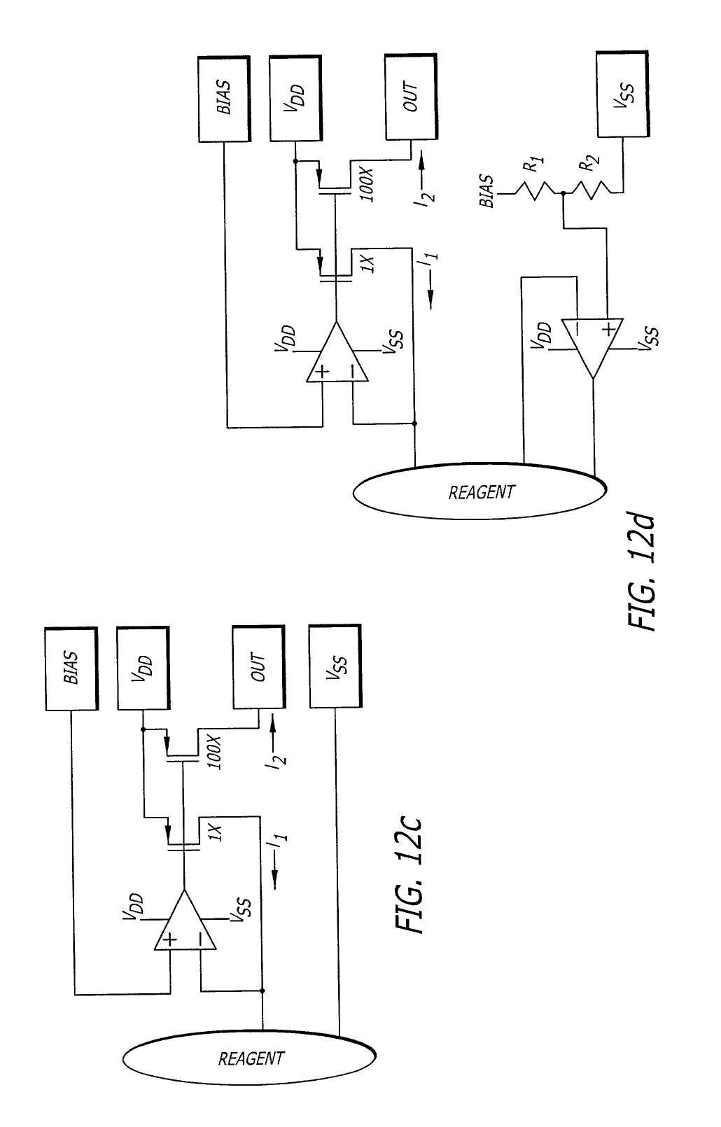

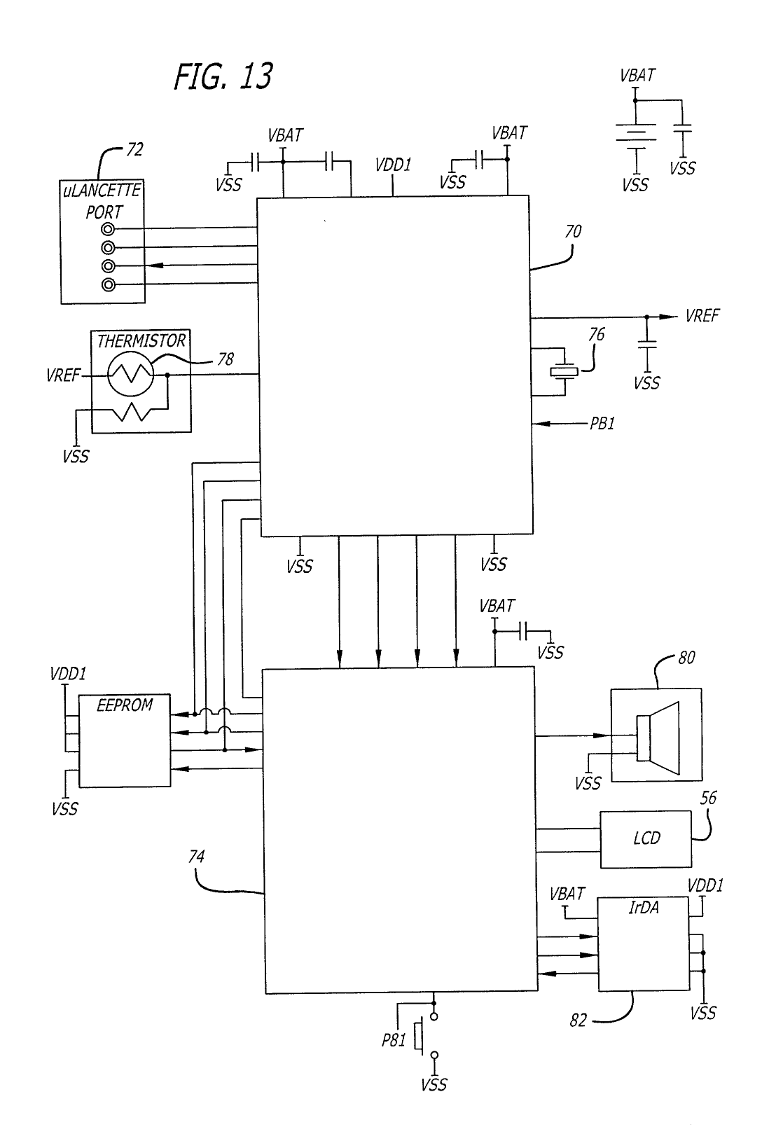

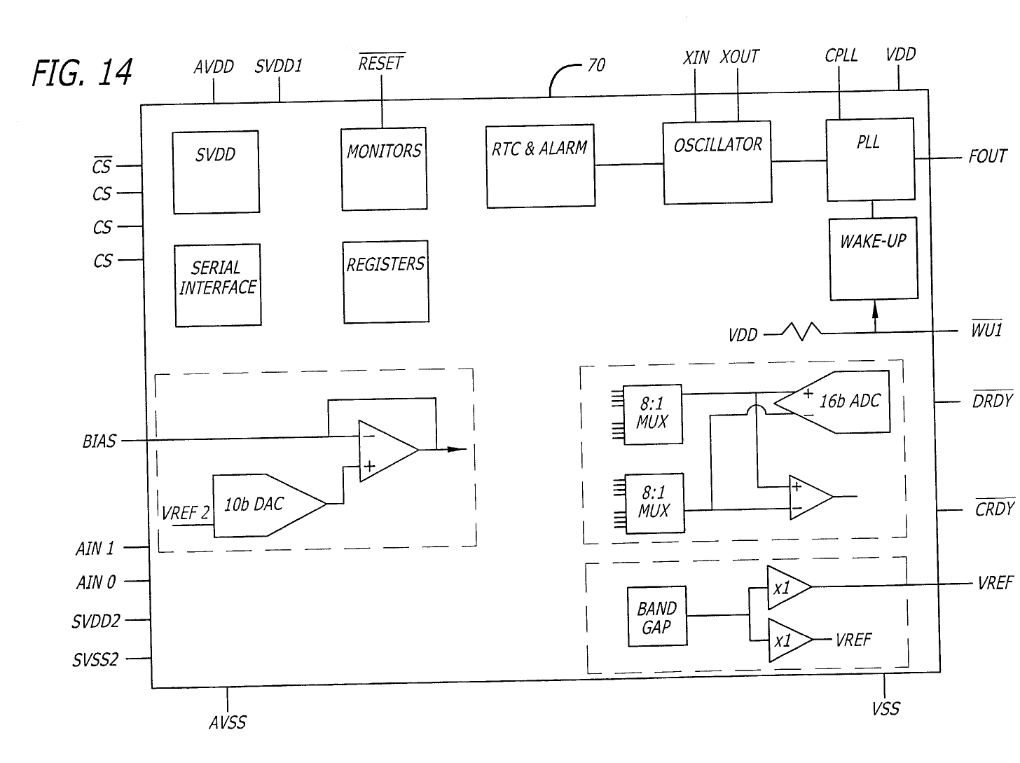

The electronic circuitry is partitioned to obtain optimal performance and cost benefit by minimizing the amount of circuitry used in the disposable probe 52. Four exemplary versions of the on-chip electronics are shown in FIGS. 12a to 12d. FIG. 12a has two electrodes for amperometric measurements at the reagent sites. A single operational amplifier amplifies the signal, with voltage levels set by an on-chip trimming network or off-chip bias. A three electrode version is shown in FIG. 12b, with an additional counter electrode bias supply. Two and three electrode versions with current mirrors are depicted in FIGS. 12c and 12d. A customized ASIC 70 (application specific integrated circuit, detailed in FIG. 14) may interface between the lancet port 72 (FIG. 13) and a microcontroller 74 for accurate portrayal and communication of the glucose levels. The ASIC contains amplification circuitry, analog-to-digital converters, a voltage reference, temperature measurement circuitry and voltage supervisory circuitry for the microcontroller. Clock functions are provided with a quartz crystal 76 and a thermistor 78 is used to provide temperature input for the compensation circuitry. An alarm 80 is included for alerting the user when a measurement is being taken. The LCD display 56 provides immediate display of the blood glucose level, whereas an external communication device such as an IrDA port 82 can be used to provide time/date stamped data for subsequent uploading into a personal computer for accurate record keeping.

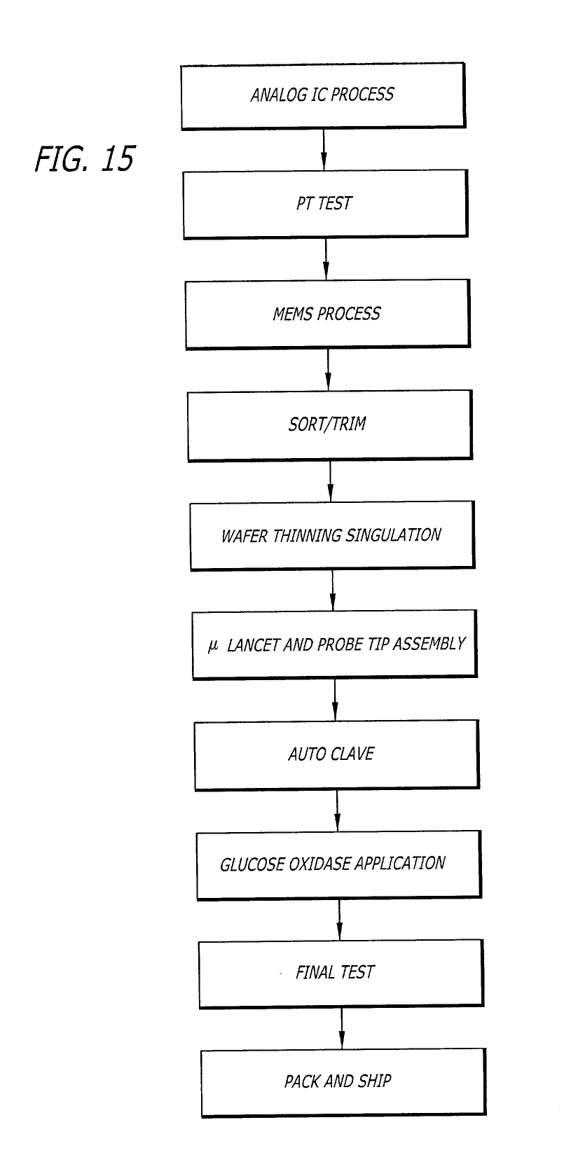

An exemplary overall process, test and assembly flow for an integrated lancet for glucose sensing is shown in FIG. 15. Starting with completion of analog IC processing of the semiconductor wafers, parameter testing (PT) is performed to measure device parameters and verify processing steps. The micromachining steps to form the recesses in the silicon wafer are performed during the MEMS processes, followed by wafer level sorting and trimming. The wafers are then thinned from the backside to reduce the thickness of the lancet and to singulate the multiplicity of devices (>60,000 on a 6″ wafer). Note that sawing is not used in this process. The micro-lancet is assembled into the probe tip assembly using adhesives, and the pads are connected to the lead frame with wirebonds followed by overmolding of the bond wires. After autoclaving, the glucose oxidase is applied by dipping or spraying, following by a drying sequence. The probe tips are placed in a specialized handler for final testing to provide verification and marking of good parts prior to final packaging and shipping.

The benefits of the present invention are good accuracy, reduced cost, disposable assembly, fast response, minimal bodily intrusion and extraction of bodily fluids, and low induced pain compared to existing methods. The device area is small for reduced cost of the silicon. Low-cost packaging and molding techniques are utilized for high volumes of the disposable device. Improved response is achieved by placing the electrodes directly in contact with subsurface epidermal layers of the skin. Extraction of bodily fluids is nearly eliminated by placing the electrodes in the tip of the lancet, considerably reducing the amount of bodily fluids drawn compared to needles. The minute size of the lancet reduces the pain of intrusion similar to that of a mosquito bite or less without concomitant itching.

While preferred embodiments of the present invention have been disclosed herein, such disclosure is only for purposes of understanding exemplary embodiments and not by way of limitation of the invention. It will be obvious skilled in the art that various changes in form and detail may be made in the invention without departing from the spirit and scope of the invention as set out in the full scope of the following claims.

[image]

What is claimed is:

1. A lancet comprising:
a semiconductor die having an integral needle-like pointed protrusion, a plurality of electrodes and a selective reagent on a surface of the protrusion and in electrical contact with at least two of the electrodes.

2. The lancet of claim 1 further comprised of a die carrier, wherein the semiconductor die and die carrier have complementary shaped regions to locate the semiconductor die within the die carrier with the protrusion on the semiconductor die extending from the die carrier.

3. The lancet of claim 2 wherein the die carrier includes a lead frame and the semiconductor die includes bonding pads electrically coupled to the electrodes, the lead frame and bonding pads being electrically coupled.

4. The lancet of claim 3 wherein the lead frame includes integral pins for electrical connection to external circuitry.

5. The lancet of claim 1 wherein the semiconductor die includes integrated signal conditioning circuitry formed therein.

6. A system for measurement of a biological quantity comprising:
a semiconductor die having an integral needle-like pointed protrusion, a plurality of electrodes and a selective reagent on a surface of the protrusion and in electrical contact with at least two of the electrodes;
a die carrier, the semiconductor die and die carrier having complementary shaped regions locating the die within the die carrier with the protrusion on the semiconductor die extending from the die carrier, the die carrier including a lead frame and the semiconductor die including bonding pads electrically coupled to the electrodes, the lead frame and bonding pads being electrically coupled by wire bonding, the lead frame including integral pins for electrical connection to external circuitry;
the semiconductor die and die carrier comprising a permanent sensor assembly;
a hand held meter body detachably connectable to the sensor assembly, the meter body comprising circuitry for processing signals from the sensor assembly and a display for displaying signal processing results.

7. The system of claim 6 wherein the circuitry for processing signals from the sensor assembly includes compensation circuitry.

8. The system of claim 6 wherein the semiconductor die includes integrated signal conditioning circuitry.

9. A system for measurement of a biological quantity comprising:
a semiconductor die having an integral needle-like pointed protrusion, a plurality of electrodes and a selective reagent on a surface of the protrusion and in electrical contact with at least two of the electrodes;
a die carrier, the semiconductor die and die carrier having complementary shaped regions locating the die within the die carrier with the protrusion on the semiconductor die extending from the die carrier, the die carrier including a lead frame and the semiconductor die including bonding pads electrically coupled to the electrodes, the lead frame and bonding pads being electrically coupled by wire bonding, the lead frame including integral pins for electrical connection to external circuitry;
the semiconductor die and die carrier comprising a permanent sensor assembly;
a hand held meter body detachably connectable to the sensor assembly, the meter body comprising circuitry for processing signals from the sensor assembly and external communication.

10. The system of claim 9 wherein the circuitry for processing signals from the sensor assembly includes compensation circuitry.

11. The system of claim 9 wherein the semiconductor die includes integrated signal conditioning circuitry.
